(19) United States
(12) Patent Application Publication
(10) Pub. No.: US 2008/0119367 A1
(43) Pub. Date: May 22, 2008

(54) Prognosis of Renal Cell Carcinoma

(75) Inventors: George Vasmatzis, Byron, MN (US); John C. Cheville, Pine Island, MN (US); Farhad Kosari, Rochester, MN (US); Alexander S. Parker, Jacksonville, FL (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 11/721,794

(22) PCT Filed: Dec. 16, 2005
(86) PCT No.: PCT/US05/45568

§ 371 Date: Jun. 14, 2007

Related U.S. Application Data
(60) Provisional application No. 60/636,920, filed on Dec. 17, 2004.

(51) Int. Cl. C40B 30/04; C12Q 1/68; G01N 33/53; C40B 40/08

(52) U.S. Cl. 506/9; 435/6; 435/7.1; 506/17

(57) ABSTRACT

Methods and materials related to determining renal cell carcinoma aggressiveness are provided. For example, methods for determining whether a mammal with renal cell carcinoma will have a good or poor outcome are provided. In addition, nucleic acid arrays that can be used to determine whether a mammal with renal cell carcinoma will have a good or poor outcome are provided.

### BACKGROUND

1. Technical Field

This document provides methods and materials related to predicting the aggressiveness of renal cell carcinoma in a mammal.

2. Background Information

The incidence and deaths caused by renal cell carcinoma (RCC) are increasing in the United States. Of particular note, incidence and mortality rates for RCC have risen steadily for more than 20 years among both genders, and these trends are not explained by the increased use of abdominal imaging (Chow et al., JAMA, 281:1628-31 (1999)). Indeed, mortality from RCC has increased over 37% since 1950. The standard and only curative treatment for RCC is surgical resection. The majority of patients with RCC confined to the kidney will be cured by surgery; however, about 30 percent of patients will develop metastases and die of RCC following removal of a confined tumor.

RCC encompasses a group of at least five subtypes with unique morphologic, genetic, and behavioral characteristics (Cheville et al., Am. J. Surg. Pathol., 27:612-24 (2003)). Cancer-specific survival is dependent on subtype, and over 80 percent of RCCs and the vast majority of RCC-related deaths are due to clear cell RCC (CRCC). To date, tumor stage and grade are the primary prognostic indicators for patients with CRCC treated by nephrectomy (Gettman et al., Cancer, 91:354-61 (2001)). There is, however, variability in patient outcome that cannot be explained by the combination of stage and grade.

### SUMMARY

This document relates to methods and materials involved in determining the aggressiveness of RCC. For example, this document provides methods and materials that can be used to determine whether a mammal (e.g., a human) having RCC (e.g., CRCC) will experience a good outcome or a poor outcome. Such materials include, without limitation, nucleic acid arrays that can be used to predict RCC aggressiveness in a mammal. These arrays can allow clinicians to predict the aggressiveness of RCC based on a determination of the expression levels of one or more nucleic acids that are differentially expressed in aggressive RCC cells as compared to non-aggressive RCC cells.

In general, this document features a method for determining whether a mammal with renal cell carcinoma will have a good or poor outcome. The good outcome can be living without recurrence of renal cell carcinoma for at least two year following treatment, and the poor outcome can be dying with renal cell carcinoma within four years of diagnosis or having metastatic renal cell carcinoma within four years of diagnosis. The method includes determining whether or not the mammal contains renal cell carcinoma cells that express SAA2, HSPC150, xs04h08.x1, IL-8, BIRC3, or CKS2 nucleic acid to an extent greater than the average level of expression exhibited in control cells, where the control cells are control renal cell carcinoma cells from a control mammal having the good outcome, where the presence of the renal cell carcinoma cells indicates that the mammal will have the poor outcome, and where the absence of the renal cell carcinoma cells indicates that the mammal will have the good outcome. The mammal can be a human. The renal cell carcinoma can be a clear cell renal cell carcinoma. The treatment can include a nephrectomy. The poor outcome can include dying with renal cell carcinoma within four years of diagnosis. The poor outcome can include having metastatic renal cell carcinoma within four years of diagnosis. The method can include determining whether or not the mammal contains renal cell carcinoma cells that express SAA2 nucleic acid to an extent greater than the average level of expression exhibited in the control cells. The method can include determining whether or not the mammal contains renal cell carcinoma cells that express xs04h08.x1 nucleic acid to an extent greater than the average level of expression exhibited in the control cells. The method can include determining whether or not the mammal contains renal cell carcinoma cells that express IL-8 nucleic acid to an extent greater than the average level of expression exhibited in the control cells. The method can include determining whether or not the mammal contains renal cell carcinoma cells that express CKS2 nucleic acid to an extent greater than the average level of expression exhibited in the control cells. The method can include determining whether or not the mammal contains renal cell carcinoma cells that express two or more of the nucleic acids selected from the group consisting of SAA2, HSPC150, xs04h08.x1, IL-8, BIRC3, and CKS2 nucleic acid to an extent greater than the average level of expression exhibited in the control cells. The method can include determining whether or not the mammal contains renal cell carcinoma cells that express three or more of the nucleic acids selected from the group consisting of SAA2, HSPC150, xs04h08.x1, IL-8, and CKS2 nucleic acid to an extent greater than the average level of expression exhibited in the control cells. The method can include determining whether or not the mammal contains renal cell carcinoma cells that express SAA2, HSPC150, xs04h08.x1, IL-8, and CKS2 nucleic acid to an extent greater than the average level of expression exhibited in the control cells. The determining step can include measuring the level of SAA2, HSPC150, xs04h08.x1, IL-8, BIRC3, or CKS2 mRNA expressed in the renal cell carcinoma cells. The determining step can include measuring the level of polypeptide expressed from SAA2, HSPC150, xs04h08.x1, IL-8, BIRC3, or CKS2 nucleic acid in the renal cell carcinoma cells.

In another embodiment, this document features a method for determining whether a mammal with renal cell carcinoma will have a good or poor outcome. The good outcome can be living without recurrence of renal cell carcinoma for at least two year following treatment, and the poor outcome can be dying with renal cell carcinoma within four years of diagnosis or having metastatic renal cell carcinoma within four years of diagnosis. The method includes determining whether or not the mammal contains renal cell carcinoma cells that express a nucleic acid selected from the group consisting of ECRG4, FLJ32535, PPP2CA, FILIP1, SDPR, SCN4B, PTPRB, 7n51g0.3.x1, TEK, SHANK3, wa07c11.x1, ARG99, SYNPO2, EMCN, DKFZp686P0921_r1, TU3A, NPY1R, MAPT, UI-H-BI4-aqb-d-08-0-UI.s1, LDB2, tn49h09.x1, PDZK3, FLJ22655, tb28a05.x1, FCN3, NX17, CUBN, EPAS1, LOC340024, PLN, ERG, DKFZP564O0823, SLC6A19, and yc17g11.s1 nucleic acid to an extent less than the average level of expression exhibited in control cells, where the control cells are control renal cell carcinoma cells from a control mammal having the good outcome, where the presence of the renal cell carcinoma cells indicates that the mammal will have the poor outcome, and where the absence of the renal cell carcinoma cells indicates that the mammal will have the good outcome. The mammal can be a human. The renal cell carcinoma can be clear cell renal cell carcinoma. The treatment can be a nephrectomy. The poor outcome can be dying with renal cell carcinoma within four years of diagnosis. The poor outcome can be having metastatic renal cell carcinoma within four years of diagnosis. The method can include determining whether or not the mammal contains renal cell carcinoma cells that express two or more of the nucleic acids selected from the group to an extent less than the average level of expression exhibited in the control cells. The method can include determining whether or not the mammal contains renal cell carcinoma cells that express three or more of the nucleic acids selected from the group to an extent less than the average level of expression exhibited in the control cells. The method can include determining whether or not the mammal contains renal cell carcinoma cells that express four or more of the nucleic acids selected from the group to an extent less than the average level of expression exhibited in the control cells. The method can include determining whether or not the mammal contains renal cell carcinoma cells that express five or more of the nucleic acids selected from the group to an extent less than the average level of expression exhibited in the control cells. The determining step can include measuring the level of ECRG4, FLJ32535, PPP2CA, FILIP1, SDPR, SCN4B, PTPRB, 7n51g0.3.x1, TEK, SHANK3, wa07c11.x1, ARG99, SYNPO2, EMCN, DKFZp686P0921_r1, TU3A, NPY1R, MAPT, UI-H-BI4-aqb-d-08-0-UI.s1, LDB2, tn49h09.x1, PDZK3, FLJ22655, tb28a05.x1, FCN3, NX17, CUBN, EPAS1, LOC340024, PLN, ERG, DKFZP564O0823, SLC6A19, or yc17g11.s1 mRNA expressed in the renal cell carcinoma cells. The determining step can include measuring the level of polypeptide expressed from ECRG4, FLJ32535, PPP2CA, FILIP1, SDPR, SCN4B, PTPRB, 7n51g0.3.x1, TEK, SHANK3, wa07c11.x1, ARG99, SYNPO2, EMCN, DKFZp686P0921_r1, TU3A, NPY1R, MAPT, UI-H-BI4-aqb-d-08-0-UI.s1, LDB2, tn49h09.x1, PDZK3, FLJ22655, tb28a05.x1, FCN3, NX17, CUBN, EPAS1, LOC340024, PLN, ERG, DKFZP564O0823, SLC6A19, or yc17g11.s1 nucleic acid in the renal cell carcinoma cells.

In another aspect, this document features a nucleic acid array containing at least five nucleic acid molecules, where each of the at least five nucleic acid molecules has a different nucleic acid sequence, and where at least 50 percent of the nucleic acid molecules of the array have a sequence from a nucleic acid selected from the group consisting of SAA2, HSPC150, xs04h08.x1, IL-8, BIRC3, CKS2, BIRC5, ECRG4, FLJ32535, PPP2CA, FILIP1, SDPR, SCN4B, PTPRB, 7n51g0.3.x1, TEK, SHANK3, wa07c11.x1, ARG99, SYNPO2, EMCN, DKFZp686P0921_r1, TU3A, NPY1R, MAPT, UI-H-BI4-aqb-d-08-0-UI.s1, LDB2, tn49h09.x1, PDZK3, FLJ22655, tb28a05.x1, FCN3, NX17, CUBN, EPAS1, LOC340024, PLN, ERG, DKFZP564O0823, SLC6A19, or yc17g11.s1. The array can contain at least ten nucleic acid molecules, wherein each of the at least ten nucleic acid molecules has a different nucleic acid sequence. The array can contain at least twenty nucleic acid molecules, wherein each of the at least twenty nucleic acid molecules has a different nucleic acid sequence. Each of the nucleic acid molecules that contain a sequence from a nucleic acid selected from the group can contain no more than three mismatches. At least 75 percent of the nucleic acid molecules of the array can contain a sequence from a nucleic acid selected from the group. At least 95 percent of the nucleic acid molecules of the array can contain a sequence from a nucleic acid selected from the group. The array can contain glass.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1(a) is a diagram depicting the unsupervised clustering of the 41 cases from the microarray data. Genes (1730) that were present in at least 50 percent of the cases and had expression levels that varied by at least 1.2 SD of log intensity unit were used. The clade on the left consists of normal samples (green legends) exclusively, the clade on the right includes two smaller clusters; a cluster on the left consisting of primary tumors in patients with poor outcome (red legends) and metastatic tumor samples (pink legends), and a cluster on the right consisting primary sample of patients with good outcome (blue legends). FIG. 1(b) is a diagram depicting the unsupervised clustering of the non-neoplastic tissues. Genes (1273) that were present in at least 50 percent of the cases and had expression levels that varied by at least 1.0 SD of log intensity unit were used. Dark and light green legends indicate non-neoplastic tissues adjacent to poor and good outcome primaries, respectively. FIG. 1(c) is a diagram depicting the unsupervised clustering of the poor outcome primary and metastasis cases. Genes (1568) that were present in at least 50 percent of the cases and had expression levels that varied by at least 1.2 SD of log intensity unit were used.

FIG. 2 is a heat map depicting the expression levels of the 34 genes selected using three algorithms. High and low expression levels are shown in red and blue colors, respectively, according to the scale at the bottom of the heat map. The red and blue bars on the left identify the up- and down-regulated genes in primary tumors with good outcome compared to the poor outcome primaries and metastatic CRCC, respectively. The dendogram on the top illustrates the supervised clustering results based on the 34 selected genes. The colors of the legends are as defined in FIG. 1.

FIG. 3 is a graph plotting the un-normalized (raw) data depicting the expression values of the four candidate normalization genes across the 55 sample validation cohort. Non-neoplastic cases adjacent to good and poor outcome primaries are depicted in light and dark green, respectively. Good outcome primaries, poor outcome primaries, and metastatic cases are represented in blue, red, and pink, respectively. GapDH and B2M display the largest standard deviations (SD) and have higher expression in CRCC tissues than in non-neoplastic samples. KPNA6 expression levels display the lowest variation and do not show differential expression between the tumors and the non-neoplastic cases.

FIG. 4 contains two graphs of quantitative RT-PCR validation results of selected candidate biomarkers. FIG. 4(a) is a graph plotting the values for 10 genes with the most significantly down-regulated expression in aggressive and metastatic CRCC compared to non-aggressive CRCC. FIG. 4(b) is a graph plotting the values for three genes with the most significantly up-regulated expression in aggressive and metastatic CRCC compared to non-aggressive CRCC. Color designations are as defined in FIG. 3.

FIG. 5 is a diagram of the quantitative RT-PCR experimental data on the 55 sample validation cohort visualized by the TREEVIEW program. Gene names are listed on the right of the heat map. The dendogram displays the clustering of the cohort by the CLUSTER program. In the map, red and green indicate expression levels higher and lower than the mean expression, respectively. The color scheme for the dendogram labels is as defined in FIG. 1.

### DETAILED DESCRIPTION

This document provides to methods and materials involved in determining the aggressiveness of RCC. For example, this document provides methods for determining whether a mammal with RCC will have a good or poor outcome. A good outcome can be an outcome where the mammal (e.g., human) lives without RCC recurrence for at least one, two, three, four, or more years following treatment for the RCC. Treatment of RCC can include surgical resection of the RCC. A poor outcome can be (1) an outcome where the mammal dies with RCC within one, two, three, four, or more years of diagnosis or (2) an outcome where the mammal experiences metastatic RCC within one, two, three, four, or more years of diagnosis. This document also provides nucleic acid arrays that can be used to determine whether a mammal with RCC will have a good or poor outcome. Such arrays can allow clinicians to determine the aggressiveness of RCC based on a determination of the expression levels of one or more nucleic acids that are differentially expressed in aggressive and non-aggressive RCC.

### 1. Determining Whether a Mammal With RCC Will Have a Good or Poor Outcome

The outcome of a mammal having RCC can be determined by assessing the expression levels of one or more nucleic acids within RCC cells. For example, the expression level of one or more (e.g., two, three, four, five, six, seven, eight, nine, ten, or more) of the following nucleic acids can be assessed: SAA2 (GenBank® Accession Number NM_{—}030754.2), xs04h08.x1 (GenBank® Accession Number AW270845), IL-8 (GenBank® Accession Number NM_{—}000584.2), CKS2 (GenBank® Accession Number NM_{—}001827.1), BIRC5 (GenBank® Accession Number NM_{—}001168.1), ECRG4 (GenBank® Accession Number AF325503.1), oc34c06.s1 (GenBank® Accession Number AA806965.1), PPP2CA (GenBank® Accession Number BF030448.1), FILIP1 (GenBank® Accession Number 30268230), SDPR (GenBank® Accession Number NM_{—}004657.3), SCN4B (GenBank® Accession Number NM_{—}174934.1), PTPRB (GenBank® Accession Number NM_{—}002837.2), 7n51g0.3.x1 (GenBank® Accession Number BF110268), TEK (GenBank® Accession Number NM_{—}000459.1), SHANK3 (GenBank® Accession Number BF439330.1), wa07c11.x1 (GenBank® Accession Number AI635774), ARG99 (GenBank® Accession Number AF319520.1), tz30b04.x1 (GenBank® Accession Number AI634580.1), EMCN (GenBank® Accession Number NM_{—}016242.2), DKFZp686P0921_r1 (GenBank® Accession Number AL703532), TU3A (GenBank® Accession Number 4886486), NPY1R (GenBank® Accession Number NM_{—}000909.4), MAPT (GenBank® Accession Number AA199717.1), UI-H-BI4-aqb-d-08-0-UI.s1 (GenBank® Accession Number BF508344), LDB2 (GenBank® Accession Number NM_{—}001290.1), tn49h09.x1 (GenBank® Accession Number AI590207), PDZK3 (GenBank® Accession Number AF338650.1), FLJ22655 (GenBank® Accession Number NM_{—}024730.2), tb28a05.x1 (GenBank® Accession Number AI307778), FCN3 (GenBank® Accession Number NM_{—}003665.2), NX17 (GenBank® Accession Number AF229179.1), CUBN (GenBank® Accession Number NM_{—}001081.2), EPAS1 (GenBank® Accession Number NM_{—}001430.3), LOC340024 (GenBank® Accession Number AI627358. 1), ERG (GenBank® Accession Number AA296657.1), HSPC150 (GenBank® Accession Number 7416119), PLN (GenBank® Accession Number NM_{—}002667.2), yc17g11.s1 (GenBank® Accession Number T70087.1), DKFZP564O0823 (GenBank® Accession Number NM_{—}015393.2), BIRC3 (GenBank® Accession Numbers NM_{—}001165 and NM_{—}182962), and SLC6A19 (GenBank® Accession Number NM_{—}001003841).

In one embodiment, the outcome of a mammal having RCC can be determined to be poor if the expression level of an SAA2, HSPC150, xs04h08.x1, IL-8, CKS2, BIRC3, or BIRC5 nucleic acid within an RCC sample is greater than the expression level (e.g., the average measured expression level) in non-aggressive RCC cells. Any method can be used to determine whether the expression level of a nucleic acid within a sample is greater than the expression level in non-aggressive RCC cells. For example, the SAA2, HSPC150, xs04h08.x1, IL-8, CKS2, BIRC3, or BIRC5 mRNA or polypeptide levels within an RCC sample from a mammal to be assessed can be measured and compared to the levels from non-aggressive RCC cells. In this case, if the sample contains a greater level of expression than that of the non-aggressive RCC cells, then the outcome of that mammal can be poor. In another example, the SAA2, HSPC150, xs04h08.x1, IL-8, CKS2, BIRC3, or BIRC5 mRNA or polypeptide levels within an RCC sample from a mammal to be assessed can be measured and compared to the levels from aggressive RCC cells. In this case, if the sample contains a similar level of expression as that of the aggressive RCC cells, then the outcome of that mammal can be poor. In yet another example, the SAA2, HSPC150, xs04h08.x1, IL-8, CKS2, BIRC3, or BIRC5 mRNA or polypeptide levels within an RCC sample from a mammal to be assessed can be measured and compared to reference levels contained, for example, on a reference chart or within a computer program. Such reference levels can be determined from results obtained from the assessment of a large number of aggressive and/or non-aggressive RCC samples.

In another embodiment, the outcome of a mammal having RCC can be determined to be poor if the expression level of an ECRG4, FLJ32535, PPP2CA, FILIP1, SDPR, SCN4B, PTPRB, 7n51g0.3.x1, TEK, SHANK3, wa07c11.x1, ARG99, SYNPO2, EMCN, DKFZp686P0921_r1, TU3A, NPY1R, MAPT, UI-H-BI4-aqb-d-08-0-UI.s1, LDB2, tn49h09.x1, PDZK3, FLJ22655, tb28a05.x1, FCN3, NX17, CUBN, EPAS1, LOC340024, PLN, ERG, DKFZP564O0823, SLC6A19, or yc17g11.s1 nucleic acid within an RCC sample is less than the expression level (e.g., the average measured expression level) in non-aggressive RCC cells. Any method can be used to determine whether the expression level of a nucleic acid within in sample is less than the expression level in non-aggressive RCC cells. For example, the ECRG4, FLJ32535, PPP2CA, FILIP1, SDPR, SCN4B, PTPRB, 7n51g0.3.x1, TEK, SHANK3, wa07c11.x1, ARG99, SYNPO2, EMCN, DKFZp686P0921_r1, TU3A, NPY1R, MAPT, UI-H-BI4-aqb-d-08-0-UI.s1, LDB2, tn49h09.x1, PDZK3, FLJ22655, tb28a05.x1, FCN3, NX17, CUBN, EPAS1, LOC340024, PLN, ERG, DKFZP564O0823, SLC6A19, or yc17g11.s1 mRNA or polypeptide levels within an RCC sample from a mammal to be assessed can be measured and compared to the levels from non-aggressive RCC cells. In this case, if the sample contains a reduced level of expression than that of the non-aggressive RCC cells, then the outcome of that mammal can be poor. In another example, the ECRG4, FLJ32535, PPP2CA, FILIP1, SDPR, SCN4B, PTPRB, 7n51g0.3.x1, TEK, SHANK3, wa07c11.x1, ARG99, SYNPO2, EMCN, DKFZp686P0921_r1, TU3A, NPY1R, MAPT, UI-H-BI4-aqb-d-08-0-UI.s1, LDB2, tn49h09.x1, PDZK3, FLJ22655, tb28a05.x1, FCN3, NX17, CUBN, EPAS1, LOC340024, PLN, ERG, DKFZP564O0823,

SLC6A19, or yc17g11.s1 mRNA or polypeptide levels within an RCC sample from a mammal to be assessed can be measured and compared to the levels from aggressive RCC cells. In this case, if the sample contains a similar level of expression as that of the aggressive RCC cells, then the outcome of that mammal can be poor. In yet another example, the ECRG4, FLJ32535, PPP2CA, FILIP1, SDPR, SCN4B, PTPRB, 7n51g0.3.x1, TEK, SHANK3, wa07c11.x1, ARG99, SYNPO2, EMCN, DKFZp686P0921_r1, TU3A, NPY1R, MAPT, UI-H-BI4-aqb-d-08-0-UI.s1, LDB2, tn49h09.x1, PDZK3, FLJ22655, tb28a05.x1, FCN3, NX17, CUBN, EPAS1, LOC340024, PLN, ERG, DKFZP564O0823, SLC6A19, or yc17g11.s1 mRNA or polypeptide levels within an RCC sample from a mammal to be assessed can be measured and compared reference levels contained, for example, on a reference chart or within a computer program. Such reference levels can be determined from results obtained from the assessment of a large number of aggressive and/or non-aggressive RCC samples.

The mammal can be any mammal such as a human, dog, cat, horse, cow, pig, goat, monkey, mouse, or rat. Any RCC cell type can be isolated and evaluated. For example, clear cell RCC cells can be isolated from a human patient and evaluated to determine if that patient contains cells that (1) express one or more nucleic acids (e.g., SAA2, HSPC150, xs04h08.x1, IL-8, CKS2, or BIRC5 nucleic acid) at a level that is greater than the expression level in non-aggressive RCC cells and/or (2) express one or more nucleic acids (e.g., ECRG4, FLJ32535, PPP2CA, FILIP1, SDPR, SCN4B, PTPRB, 7n51g0.3.x1, TEK, SHANK3, wa07c11.x1, ARG99, SYNPO2, EMCN, DKFZp686P0921_r1, TU3A, NPY1R, MAPT, UI-H-BI4-aqb-d-08-0-UI.s1, LDB2, tn49h09.x1, PDZK3, FLJ22655, tb28a05.x1, FCN3, NX17, CUBN, EPAS1, LOC340024, PLN, ERG, DKFZP564O0823, SLC6A19, or yc17g11.s1 nucleic acid) at a level that is less than the expression level in non-aggressive RCC cells.

The expression levels of any number of nucleic acids can be evaluated to determine a mammal's outcome. For example, the expression level of one or more than one (e.g., two, three, four, five, six, seven, eight, nine, ten, 15, 20, 25, 30, or more than 30) of the following nucleic acids can be used: SAA2, HSPC150, xs04h08.x1, IL-8, CKS2, BIRC5, ECRG4, FLJ32535, PPP2CA, FILIP1, SDPR, SCN4B, PTPRB, 7n51g0.3.x1, TEK, SHANK3, wa07c11.x1, ARG99, SYNPO2, EMCN, DKFZp686P0921_r1, TU3A, NPY1R, MAPT, UI-H-BI4-aqb-d-08-0-UI.s1, LDB2, tn49h09.x1, PDZK3, FLJ22655, tb28a05.x1, FCN3, NX17, CUBN, EPAS1, LOC340024, PLN, ERG, DKFZP564O0823, SLC6A19, BIRC3, or yc17g11.s1 nucleic acid. Examples of nucleic acid combinations that can be evaluated include, without limitation, NPY1R and ECRG4; EMCN and 7n51g0.3.x1; SAA2 and ECRG4; SAA2, BIRC5, and TEK; SHANK3, ARG99, SAA2, and BIRC5; and SDPR, EMCN, SAA2, and BIRC5.

A nucleic acid can be determined to be expressed at a level that is greater than or less than the expression level (e.g., average measured expression level) in non-aggressive RCC cells if the expression levels differ by at least 1 fold (e.g., 1, 2, 3, 4, 5, 6, 7, 8, 9, 10, or more fold up or down). In some embodiments, a nucleic acid is determined to be expressed at a level that is greater than or less than the expression level (e.g., average measured expression level) in non-aggressive RCC cells if the expression levels differ by at least 4 fold, either 4 fold up or 4 fold down. In addition, the non-aggressive RCC cells typically are the same type of cells as those isolated from the mammal being evaluated. In addition, the non-aggressive RCC cells (e.g., clear cell RCC cells) can be isolated from one or more mammals that are from the same species as the mammal being evaluated. Any number of mammals can be used to obtain non-aggressive RCC cells. For example, non-aggressive RCC cells can be obtained from one or more mammals (e.g., at least 5, at least 10, at least 15, at least 20, or more than 20 mammals).

Any method can be used to determine whether or not a nucleic acid is expressed at a level that is greater or less than the expression level in non-aggressive RCC cells. For example, the level of expression from a particular nucleic acid can be measured by assessing the level of mRNA expression from the nucleic acid. Levels of mRNA expression can be evaluated using, without limitation, northern blotting, slot blotting, quantitative reverse transcriptase polymerase chain reaction (RT-PCR), or chip hybridization techniques. Methods for chip hybridization assays include, without limitation, those described herein. Such methods can be used to determine simultaneously the relative expression levels of multiple mRNAs. Alternatively, the level of expression from a particular nucleic acid can be measured by assessing polypeptide levels. Polypeptide levels can be measured using any method such as immuno-based assays (e.g., ELISA), western blotting, or silver staining.

In some embodiments, polypeptide levels can be measured from a fluid sample (e.g., a serum or urine sample) to determine whether a mammal contains aggressive RCC cells. For example, the level of an FCN3, CUBN, IL8, or SAA2 polypeptide in a serum or urine sample obtained from a mammal (e.g., a human) can be measured. If the sample contains a polypeptide (e.g., IL8 or SAA2) at a level that is greater than the level in normal mammals or mammals having non-aggressive RCC cells, than that sample can be classified as coming from a mammal having aggressive RCC cells. If the sample contains a polypeptide (e.g., FCN3 or CUBN) at a level that is less than the level in normal mammals or mammals having non-aggressive RCC cells, than that sample can be classified as coming from a mammal having aggressive RCC cells.

### 2. Arrays

This document also provides nucleic acid arrays. The arrays provided herein can be two-dimensional arrays, and can contain at least 10 different nucleic acid molecules (e.g., at least 20, at least 30, at least 50, at least 100, or at least 200 different nucleic acid molecules). Each nucleic acid molecule can have any length. For example, each nucleic acid molecule can be between 10 and 250 nucleotides (e.g., between 12 and 200, 14 and 175, 15 and 150, 16 and 125, 18 and 100, 20 and 75, or 25 and 50 nucleotides) in length. In addition, each nucleic acid molecule can have any sequence. For example, the nucleic acid molecules of the arrays provided herein can contain sequences that are present within the nucleic acids listed in Table 1.

Typically, at least 25 percent (e.g., at least 30 percent, at least 40 percent, at least 50 percent, at least 60 percent, at least 75 percent, at least 80 percent, at least 85 percent, at least 90 percent, at least 95 percent, or 100 percent) of the nucleic acid molecules of an array provided herein contain a sequence that is (1) at least 10 nucleotides (e.g., at least 11, 12, 13, 14, 15, 16, 17, 18, 19, 20, 25, or more nucleotides) in length and (2) at least about 95 percent (e.g., at least about 96, 97, 98, 99, or 100) percent identical, over that length, to a sequence present within a nucleic acid listed in Table 1. For example, an array can contain 25 nucleic acid molecules located in known positions, where each of the 25 nucleic acid molecules is 100 nucleotides in length while containing a sequence that is (1) 30 nucleotides is length, and (2) 100 percent identical, over that 30 nucleotide length, to a sequence of one of the nucleic acids listed in Table 1. A nucleic acid molecule of an array provided herein can contain a sequence present within a nucleic acid listed in Table 1, where that sequence contains one or more (e.g., one, two, three, four, or more) mismatches.

The nucleic acid arrays provided herein can contain nucleic acid molecules attached to any suitable surface (e.g., plastic or glass). In addition, any method can be use to make a nucleic acid array. For example, spotting techniques and in situ synthesis techniques can be used to make nucleic acid arrays. Further, the methods disclosed in U.S. Pat. Nos. 5,744,305 and 5,143,854 can be used to make nucleic acid arrays.

The invention will be further described in the following examples, which do not limit the scope of the invention described in the claims.

### EXAMPLES

### Example 1

Prognostic Signature for Aggressive Renal Cell Carcinoma

The following experiments were performed to identify potential prognostic biomarkers predictive of aggressive CRCC.

### Patient and Tissue Selection

CRCC tumor and non-neoplastic kidney samples were selected from the Mayo Clinic RCC Biospecimens Resource directed by the Departments of Urology, Pathology and Health Sciences Research. As part of this resource, fresh non-neoplastic and neoplastic samples were collected and snap frozen from every patient undergoing nephrectomy for a renal mass. From this resource, the following groups were selected for the oligonucleotide microarray experiments: 11 primary tumor samples from patients who were still alive without disease for at least two years following nephrectomy (an example of a good outcome or non-aggressive RCC) and 9 tumors from patients with CRCC who were alive with metastatic disease or had died as a result of disease within 4 years of diagnosis (an example of a poor outcome or aggressive RCC). Since follow-up time was short for patients defined as good outcome, the SSIGN score prediction model was utilized to identify patients that had scores less than or equal to 2 and a predicted 5-year cancer-specific survival in excess of 90 percent (Frank et al., J. Urol., 168:2395-400 (2002)). The SSIGN score uses the clinicopathologic characteristics predictive of cancer-specific outcome in CRCC; namely tumor size, TNM stage, nuclear grade, and tumor necrosis. Nine CRCC metastatic tumors and 12 non-neoplastic samples were also studied. The metastatic tumor specimens included four cases that were matched with primary poor outcome CRCC.

A separate cohort of patient tumor samples was identified for validation by quantitative RT-PCR using the same criteria for good and poor outcome as used for the microarray experiments. This validation cohort consisted of 14 patients with good outcome, 17 patients with poor outcome, and nine metastatic samples. Also included in the validation study were 15 samples of adjacent non-neoplastic tissue from eight cases with good outcome and seven cases with poor outcome. Prior to all experiments, hematoxylin and eosin (H&E) stained sections from frozen tissue blocks were reviewed by a urologic pathologist with expertise in renal neoplasia to insure appropriate tissue diagnosis as well as quality and quantity of the tumor samples. Frozen tissue sections were also reviewed for pathologic features predictive of outcome (nuclear grade and necrosis). Because CRCC exhibits considerable heterogeneity in these pathologic features, and aggressive behavior is dependent on the presence of only a very small amount of the highest grade component (Lohse et al., Am. J. Clin. Pathol., 118:877-86 (2002)), tumor blocks were selected to insure that aggressive CRCC samples were predominantly high-grade (nuclear grade 3 and 4), and non-aggressive CRCC were all low-grade (nuclear grade 1 and 2). In tumor blocks, all non-neoplastic tissue was removed from the frozen block. At the end of processing, another H&E section was prepared to insure tumor quality and quantity.

### Oligonucleotide Microarray Experiments

Thirty mm³ of each tissue were sectioned at 20 or 35 μm, collected in buffer RLT (Qiagen, Valencia, Calif.) supplemented with β-mercaptoethanol and homogenized using a PT 1200C (Kinematica AG, Luzerne, Switzerland) rotor/stator homogenizer. Total RNA was isolated using the RNeasy kit (Qiagen) following manufacturer's specifications. Quality and quantity of RNA samples were analyzed by spectrophotometry and Agilent 2100 Bioanalyzer. Hybridization, washes, and scanning were performed following manufacture's protocols (Affymetrix Corp., Santa Clara, Calif.). Microarray experiments were carried out using the U133 Plus2 chipset.

### Microarray Data Analysis

Affymetrix microarray analysis software GCOS was used to process scanned chip images. The software generates a cell intensity file for each chip, which contains a single intensity value for each probe cell (.CEL file). DChip 1.3 was used to calculate Model Based Expression Index (MBEI) after data from all chips were normalized against an array with median overall intensity using invariant set method (Li and Wong, Proc. Natl. Acad. Sci., 98:31-6 (2001)). MBEI was calculated using Perfect Match/Mismatch (PM/MM) models with outlier detection and correction, and the calculated expression values were log 2 transformed. To identify differentially expressed genes in good and poor outcome cases, three algorithms were used. First, using the dChip program, probesets with a difference of 2.2 on the log scale (>4.5 fold change) between the average expression levels of the good and poor outcome cases and a p value less than 0.001 were identified (130 genes, List 1). To estimate the number of false positives in this list, the 29 cases were randomly assigned to two groups 1000 times, and the same criteria were applied to identify differentially expressed genes. The median false discovery rate (FDR) by this process was 0.8% (1 gene) and a 90th percentile of 2.3% (3 genes). Second, expression values of probesets (11,715) determined by the dChip program to be most variable across the good and poor outcome cases were exported to GeneCluster 2.0 (Whitehead/MIT for Genome Research) to identify 125 probesets with highest signal to noise ratios (List 2). The signal to noise ratio estimate, also referred to as the discriminate score (Takahashi et al., Proc. Natl. Acad. Sci., 98:9754-9 (2001)), was computed as SNR=(μ₁−μ₂)/(σ₁+σ₂), where μ and σ refer to the mean and standard deviations, respectively. A high SNR typically suggests that the expression levels of a gene display a much larger variation between the two groups compared to the variation within each group. Finally, probeset expression levels (54,607) from dChip were imported to the Prediction Analysis of Microarray (PAM) algorithm to identify 120 genes that best distinguish good and poor outcome cases (list 3). PAM uses the “shrunken centroid” approach to reduce the effects of “noisy” genes (Tibshirani et al., Proc. Natl. Acad. Sci., 99:6567-72 (2002)). The threshold for shrinking the centroids was set at 3.75.

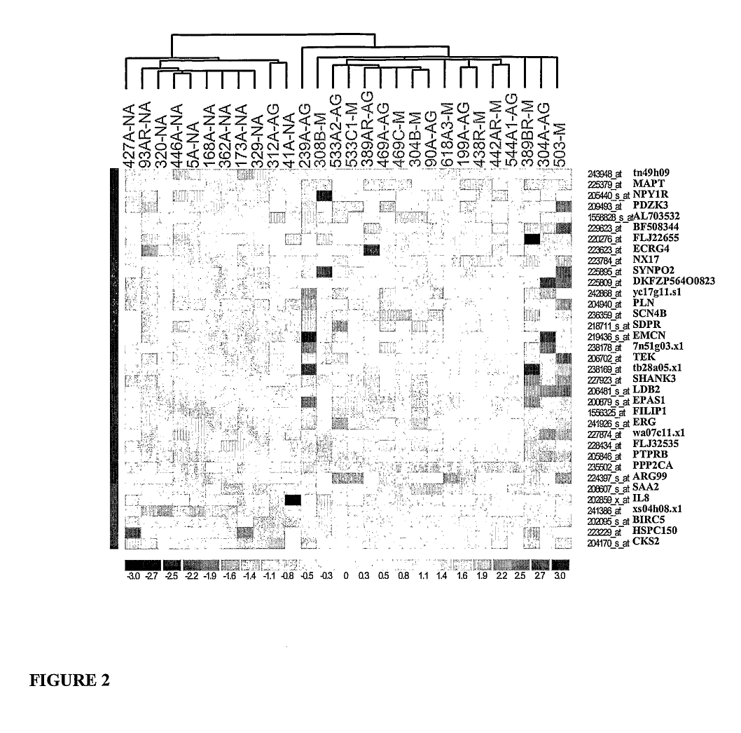

Probesets common to the three lists were identified. From this list, candidates with more than 35% absent calls in the group determined to over-express the gene were discarded. Finally, the redundant probesets representing a gene were removed. The final list included 34 probesets. This list was used for supervised clustering in the dChip program using the centroid linkage method and Euclidean distance metric (FIG. 2).

### Quantitative RT-PCR

Validation experiments were performed using tissue obtained from an independent cohort from the RCC Biospecimens Resource. Total RNA isolation and DNase treatment were carried out using RNeasy Mini kit and RNase-Free DNase Set (Qiagen) following manufacturer's specifications. RNA integrity was assessed using the Agilent 2100 Bioanalyzer.

One hundred and sixty nanograms of total RNA as measured by spectrophotometry (Nanodrop, Wilmington, Del.) were used in reverse transcription using Superscript III reverse transcriptase enzyme (Invitrogen, Carlsbad, Calif.) following manufacturer's protocol.

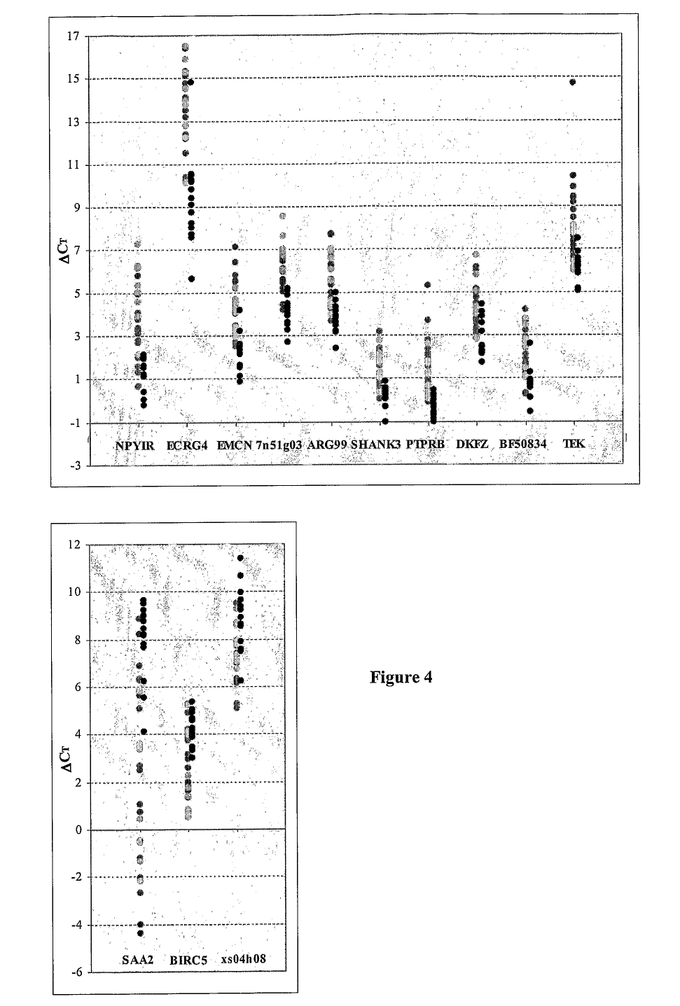

Quantitative RT-PCR experiments were performed on ABI 7900 HT system (Applied Biosystems, Foster City, Calif.). For each primer set, the optimum primer concentration (typically 0.15 nM final concentration) was determined, and standard curves were generated using a pooled cDNA sample from the validation cohort at 4-5 dilutions. Typical standard curve included 4 ng, 1 ng, 0.25 ng, 0.0625 ng, 0.0156 ng, and 0 ng (no template control) of total RNA equivalents of cDNA. To confirm that the amplification occurred on the target sequences, the amplicons were analyzed by gel electrophoresis, and the dissociation curves were examined for the presence of a single sharp peak at the melting temperature of the amplicon. The expression level of each gene was normalized by karyopherin alpha 6 (KPNA6) as: ΔC_{T}=C_{T-KPNA6}−C_{T-gene}, where C_{T} is the threshold cycle in the quantitative PCR experiment. To select the most significantly differentially expressed genes (FIG. 4), the z-score from the Mann-Whitney test was used (see, e.g., “http” colon, backslash, backslash “faculty” dot “vassar” dot “edu” backslash “lowry” backslash “utest” dot “html”).

### Clustering Analysis of the Quantitative RT-PCR Data

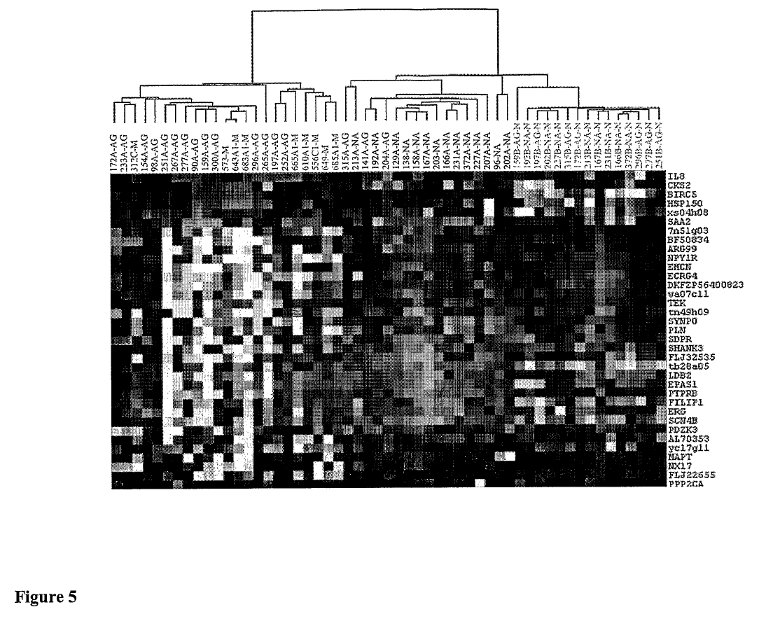

Expression levels of genes measured by quantitative PCR were first normalized by KPNA6 and then imported in the CLUSTER program. In the CLUSTER program, gene expression levels were mean centered and then scaled (normalized) such that for each gene, the sum of the squares of the values across all samples was set to one. Next, genes and samples were clustered using centroid similarity metric and average linkage clustering method. Finally, TREEVIEW program was used to visualize the results (FIG. 5).

### Clustering of Cases Based on the Overall Gene Expression Profiles

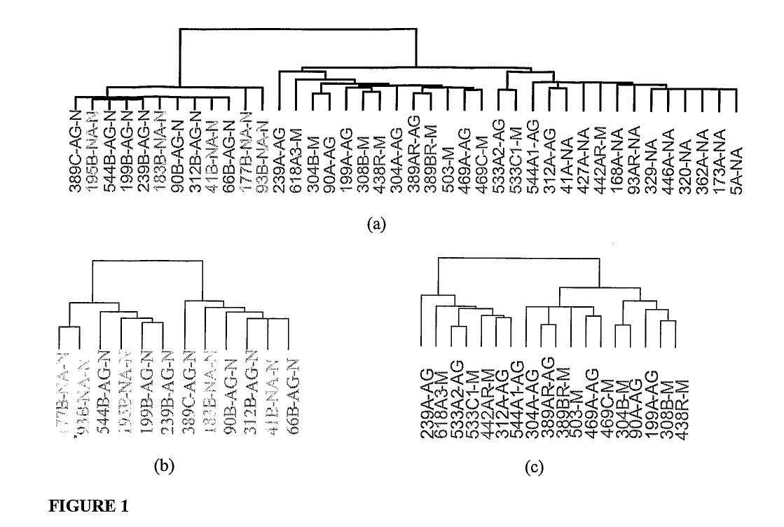

The following was performed to determine if the overall gene expression profiles can classify the cases in the microarray study. Genes with variable expression across the samples (standard deviation, SD>1.2 log intensity units and >50% present calls, 1730 probesets) were selected for unsupervised clustering (FIG. 1a). This analysis identified two major clades. One clade included all of the non-neoplastic cases from patients with non-aggressive and aggressive CRCC, and the other clade included all the cases of CRCC. This indicated that the gene expression profile common to all CRCC is significantly different from the expression profile in non-neoplastic renal tissue. The clade that included the CRCC cases consisted of two smaller clades. One clade included only the tumors from patients with poor outcome and the metastatic tumor samples. The other clade included all tumor samples from patients with good outcome, three cases from the poor outcome group, and two metastatic tumor samples. This distribution of the cohort suggests that gene expression profiles can stratify the majority of patients into appropriate outcome categories.

### Comparison of the Expression Profiles of the Non-Neoplastic Tissues Adjacent to the Good and Poor Outcome Cases

The following was performed to determine if the expression profile of the non-neoplastic kidney can determine the aggressive behavior of CRCC. In the overall unsupervised clustering plot (FIG. 1a), the non-neoplastic cases adjacent to the poor and good outcome cases were interspersed. To insure that the clustering pattern of the non-neoplastic cases was not influenced by the CRCC expression profiles, the non-neoplastic cases were examined separately. Genes with variable expression (SD>1.0 log intensity units, >50% present, 1273 probesets) were identified for unsupervised clustering (FIG. 1b). Again, the non-neoplastic tissues from patients with good and poor outcome did not separate into distinct clusters; the five matched non-neoplastic kidney samples from patients with good outcome were interspersed among the seven non-neoplastic samples from patients with poor outcome. The expression profiles of the two groups were also compared; but did not identify any significantly differentially expressed genes. These analyses suggest that the gene expression in the non-neoplastic kidney is not associated with the behavior of CRCC.

### Comparison of the Expression Profiles of the Poor Outcome CRCC Primary and the CRCC Metastatic Cases

Expression profiles were examined to determine if the profiles could discriminate poor outcome primary CRCC from the metastatic CRCC. In the overall unsupervised clustering (FIG. 1a), the poor outcome primaries and CRCC metastasis cases were interspersed. To insure that the clustering pattern was not influenced by the expression profiles of the non-neoplastic and the good outcome cases, expression profiles of the tumor samples from poor outcome and metastatic samples were analyzed separately. Genes with most variable expression across the two groups (SD>1.2 log intensity units; >50% present calls, 1568 probesets) were selected for unsupervised clustering (FIG. 1c). Here again, the poor outcome cases were interspersed evenly among the metastatic tumor samples. The expression profiles of the two groups were compared using the dChip and PAM algorithms. By the dChip algorithm, the number of differentially expressed genes between the two groups was comparable to the number of differentially expressed genes found by randomly assigning the metastatic samples and the poor outcome primaries to two groups. The median false discovery rate (FDR) was ˜100% and the 90^{th} percentile FDR was 300-400%, depending on the significance criteria. PAM was used to identify a group of genes that can be used for classification of poor outcome primary and CRCC metastasis cases. The average misclassification error with any possible threshold for “shrinking centroids” was 40-60 percent, suggesting that there were no set of genes that could correctly classify metastatic and poor outcome primaries in two groups.

### Comparison of Expression Profiles of CRCC With Different Outcome

Since the primary tumors associated with poor outcome and the metastatic samples showed similar expression profiles, metastatic tumor samples and primary tumors with poor outcome were grouped together and compared with primary tumors with good outcome. This increased the statistical power for identification of significantly differentially expressed genes.

To identify probesets that are most relevant to CRCC outcome, the signal to noise selection criteria and the PAM algorithm were used in addition to the fold change and p value criteria provided by the dChip software. In each case, comparisons were made for the gene expression values in the primary CRCC with good outcome versus the primary CRCC with poor outcome and metastatic samples. The top 120 to 130 candidate prognostic biomarkers were selected using the three statistical algorithms. 130 probesets that displayed a fold change of at least 4.5 and p<0.001 (median FDR=0.8% and 90 percentile FDR=2.3%) by dChip were identified. In addition, 125 probesets with highest signal to noise ratio by GeneCluster and 120 probesets by PAM after the centroids were “shrunken” by a factor of 3.75 were identified. With the results from these three selection methods, probesets common in the three lists that also had a present (P) call by the dChip algorithm in at least 65 percent of the cases determined to over-express the gene were selected. Finally, multiple probesets representing the same gene were discarded so that the listing would represent unique individual gene expressions. The final candidate list included 34 probesets corresponding to 34 unique transcripts (Table 1). The majority of the 34 candidate biomarkers identified by this analysis (29 of 34; 85%) displayed down regulation of expression in the aggressive CRCC compared to the non-aggressive CRCC.

TABLE 1
Candidate biomarkers predictive of CRCC outcome.

| Gene Id | dChip-R | PAM-R | SNR-R | TotalRank | Description |
|---|---|---|---|---|---|
| FLJ32535 | 12 | 1 | 27 | 40 | butyrophilin 3, oc 34c06.s1 |
| BIRC5 | 21 | 18 | 1 | 40 | Baculoviral IAP rep-cont. 5 |
| | | | | | (survivin) |
| PPP2CA | 25 | 14 | 2 | 41 | protein phosphatase 2 (formerly 2 |
| | | | | | catalytic subunit, alpha isoform |
| xs04h08.x1 | 31 | 6 | 5 | 42 | Null (GenBank ® Accession Number |
| | | | | | AW270845) |
| ECRG4 | 46 | 4 | 3 | 53 | esophageal cancer related gene 4 |
| | | | | | protein |
| FILIP1 | 20 | 21 | 18 | 59 | filamin A interacting protein 1 |
| EPAS1 | 1 | 8 | 60 | 69 | endothelial PAS domain protein 1 |
| SCN4B | 34 | 38 | 7 | 79 | sodium channel, voltage-gated, |
| | | | | | type IV, beta |
| PTPRB | 6 | 2 | 72 | 80 | protein tyrosine phosphatase, |
| | | | | | receptor type, B |
| SDPR | 23 | 11 | 47 | 81 | serum deprivation response |
| | | | | | (phosphatidylserine binding prote |
| 7n51g03.x1 | 19 | 37 | 34 | 90 | Null (GenBank ® Accession |
| | | | | | Number BF110268) |
| SHANK3 | 11 | 13 | 73 | 97 | SH3 and multiple ankyrin repeat |
| | | | | | domains 3 |
| EMCN | 33 | 49 | 26 | 108 | endomucin |
| ARG99 | 38 | 62 | 10 | 110 | ARG99 protein |
| TEK | 39 | 30 | 42 | 111 | TEK tyrosine kinase, endothelial |
| | | | | | (venous malformations, multiple |
| | | | | | cutaneous and mucosal) |
| SYNPO2 | 28 | 41 | 45 | 114 | synaptopodin 2 |
| wa07c11.x1 | 10 | 7 | 99 | 116 | Null (GenBank ® Accession |
| | | | | | Number AI635774) |
| AL703532 | 43 | 75 | 8 | 126 | null |
| SAA2 | 56 | 60 | 15 | 131 | serum amyloid A2 |
| MAPT | 47 | 73 | 13 | 133 | microtubule-associated protein tau |
| HSPC150 | 42 | 81 | 28 | 151 | HSPC150 protein similar to |
| | | | | | ubiquitin-conjugating enzyme |
| PLN | 63 | 50 | 38 | 151 | phospholamban |
| PDZK3 | 36 | 63 | 53 | 152 | PDZ domain containing 3 |
| ERG | 14 | 46 | 100 | 160 | v-ets erythroblastosis virus E26 |
| | | | | | oncogene like (avian) |
| CKS2 | 15 | 52 | 93 | 160 | CDC28 protein kinase regulatory |
| | | | | | subunit 2 |
| IL8 | 99 | 42 | 23 | 164 | interleukin 8 |
| tb28a05.x1 | 40 | 98 | 35 | 173 | tb28a05.x1 (GenBank ® |
| | | | | | Accession Number AI307778) |
| LDB2 | 16 | 44 | 115 | 175 | LIM domain binding 2 |
| DKFZP564O0823 | 74 | 68 | 36 | 178 | DKFZP564O0823 protein, |
| | | | | | tu03g12.x1 |
| NPY1R | 35 | 36 | 109 | 180 | neuropeptide Y receptor Y1 |
| BF508344 | 18 | 65 | 112 | 195 | null |
| FLJ22655 | 80 | 105 | 14 | 199 | hypothetical protein FLJ22655 |
| yc17g11.s1 | 32 | 79 | 89 | 200 | null |
| NX17 | 37 | 76 | 91 | 204 | kidney-specific membrane protein |

dChip-R, PAM-R, and SNR-R denote the rankings by dChip (based on fold change and p value), PAM, and signal to noise ratio, respectively. TotalRank denotes the sum of the three rankings. Up-regulated genes in poor outcome primary and metastatic CRCC compared to good outcome primaries are denoted in bold letters.

With this set of differentially expressed targets, hierarchical clustering of the 29 CRCC tissues was repeated based on the newly identified 34 probesets. From this analysis, clustering trees were produced that revealed two major subgroups. One subgroup contained all 18 (100 percent) of aggressive CRCC and metastatic CRCC samples and one case of the non-aggressive CRCC. The other subgroup included 91 percent (10 of 11) of the tissues from the non-aggressive CRCCs (FIG. 2).

### Validation by Quantitative RT-PCR

The results from the gene array experiments were validated by examining the expression of the 34 candidate biomarkers in an independent cohort of CRCC samples using a quantitative RT-PCR assay. Compared to the microarray technology, the quantitative RT-PCR technique provides a much wider dynamic range (5-6 orders of magnitude) and thus a more accurate means of measuring relative expression values of genes.

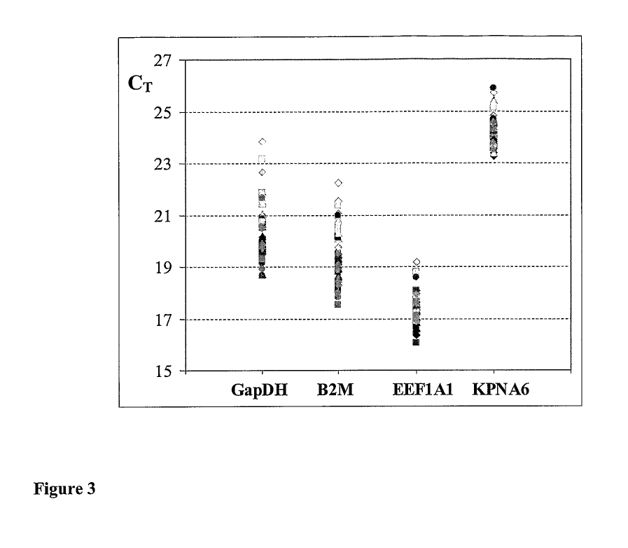

Before proceeding with the validation, genes that could be used for normalization of expression levels of samples were first identified from the microarray data. Two genes, eukaryotic translation elongation factor 1 alpha 1 (EEF1A1) and karyopherin alpha 6 (KPNA6), were selected from among the five genes with the lowest expression standard deviations in the microarray data. In addition, two common genes, beta-2-microglobin (B2M), and glyceraldehyde 3-phosphate dehydrogenase (GapDH), were examined. The expression levels of all four genes were measured by quantitative RT-PCR (FIG. 3). As expected from the microarray data, GapDH displayed the highest variation across the samples, followed by B2M. On the other hand, KPNA6 displayed the least variation across all samples. More importantly, expression of GapDH (and B2M) was lower in non-neoplastic kidney than in the RCC cases (p<1.0×10⁻⁵ for both genes). On the contrary, KPNA6 expression was not statistically different among the CRCC and non-neoplastic tissues. Furthermore, expression levels of KPNA6 in the samples were comparable to the expression levels of most of the candidate biomarkers and on average 10-20 fold (approximately 4 cycles in a quantitative PCR experiment) lower than the expression levels of GapDH. Thus, KPNA6 was selected for normalization of the quantitative PCR data.

The expression levels of the 34 transcripts were measured across the validation cohort. All of the candidate biomarkers, except IL-8, displayed significant differential expression by quantitative RT-PCR (p<0.001 for 28 candidates and p<0.005 for the remaining 5 candidates), as predicted by the microarray analysis. In the microarray experiments, IL-8 expression was up-regulated in poor outcome primary and metastatic CRCC relative to good outcome primaries. In the validation cohort, the up-regulation of IL-8 in poor outcome primaries and metastatic CRCC cases was marginal (p<0.055). FIG. 4 (top panel) illustrates expression levels of 10 candidate biomarkers that were most significantly down-regulated in aggressive and metastatic CRCC compared to non-aggressive CRCC by the Mann-Whitney test, while FIG. 4 (bottom panel) illustrates 3 candidate biomarkers that showed the highest level of up-regulation in aggressive and metastatic CRCC compared to non-aggressive CRCC. Of note, every cycle difference in these experiments represents about 2 fold differential expression. For example, for ECRG4, a difference of more than 4 cycles in the mean expression levels between the non-aggressive CRCCs and aggressive CRCCs was detected, indicating an about 15 fold difference in expression levels between the two groups.

Hierarchical clustering of the quantitative RT-PCR data confirmed that the 34 genes selected from the gene chip arrays had prognostic significance for CRCC (FIG. 5). As the figure shows, there were two main subgroups identified in the validation cohort. One subgroup included 23 of 26 (88 percent) of the aggressive and metastatic CRCC cases and one case of the non-aggressive CRCC. The other main subgroup included two further clusters, one containing all 15 (100 percent) of the non-neoplastic tissues and the other containing 13 of the 14 (93 percent) non-aggressive cases and the remaining 3 cases of aggressive primaries.

Two additional genes, baculoviral IAP repeat-containing 3 (BIRC3; GenBank accession numbers NM_{—}001165 and NM_{—}182962) and solute carrier family 6 (neutral amino acid transporter), member 19 (SLC6A19; GenBank accession number NM_{—}001003841), were identified as candidate biomarkers predictive of CRCC outcome using the microarray data analysis described herein. In addition, both BIRC3 (up regulated in aggressive CRCC; p value on the independent sample 0.0051) and SLC6A19 (down regulated in aggressive CRCC; p-value on independent sample=0.00031) were validated using the quantitative RT-PCR procedures described herein.

In summary, using genomic profiling and quantitative RT-PCR validation on tissue samples from two well-characterized cohorts of CRCC patients, a panel of genes that were differentially expressed between patients with good and poor outcome was identified. Unsupervised clustering techniques using data from the oligonucleotide microarray experiments separated the CRCC samples into their respective outcome categories indicating unique gene expression profiles predictive of patient outcome. The results revealed that there was no difference in the gene expression profile between normal kidney from patients with aggressive and non-aggressive CRCC, suggesting that the transcriptional profile of the non-involved kidney does not influence the outcome of the tumor. Additionally, primary CRCC with aggressive behavior did not exhibit a significantly different gene expression profile from metastatic samples. This observation suggests that gene expression alterations that result in aggressive behavior and metastatic potential can be identified in the primary tumor. However, it could not be determined if the key and perhaps subtle changes in the expression profile that are needed for metastasis are present in the primary. In subsequent analyses, 34 unique transcripts whose expression values differed significantly between non-aggressive and aggressive CRCC were identified. Validation studies using quantitative RT-PCR on an independent set of tissues confirmed the oligonucleotide microarray experiments and further supported this set of genes as potential biomarkers for CRCC aggressiveness and patient outcome.

The use of non-aggressive and aggressive CRCC including metastatic CRCC samples allowed for the identification of a genetic profile indicative of tumor aggressiveness. There were a number of genes that showed increased expression in aggressive CRCC as compared to non-aggressive tumors that are of note. Survivin (BIRC5) is a member of the inhibitor of apoptosis protein family, and its expression both at the mRNA and protein level is associated with more aggressive behavior in carcinomas of the larynx, liver, prostate, lung, ovary, stomach and others (Kren et al., Appl. Immunohistochem. Mol. Morphol., 12:44-9 (2004); Pizem et al., Histopathology, 45:180-6 (2004); Shariat et al., Cancer, 100:751-7 (2004); and Miyachi et al., Gastric Cancer, 6:217-24 (2003)). The results provided herein, however, demonstrate an association between survivin mRNA expression and CRCC aggressiveness.

Interleukin 8 (IL-8), a potent chemotactic cytokine for inflammatory cells, exhibited higher expression levels in the aggressive compared to the non-aggressive CRCCs by the microarray data. Interleukin 8 is implicated in the migration of lymphocytes into tumors through an alpha-1 integrin mediated pathway in the extracellular matrix, and studies demonstrate that neutralizing antisera specific to IL-8 inhibit tumor-infiltrating lymphocyte migration (Ferrero et al., Eur. J. Immunol., 28:2530-6 (1998)). It is of note that this differential expression was marginally significant (p<0.055) by the RT-PCR experiments. Another gene, serum amyloid A, has been identified in the serum of CRCC patients, and elevated serum levels are associated with aggressive CRCC (Kimura et al., Cancer, 92:2072-5 (2001)). Serum amyloid A1 and A2 are acute phase reactants whose expression is regulated in part by interleukin 1 and 6 (Glojnaric et al., Clin. Chem. Lab. Med., 39:129-33 (2001); Blay et al., Int. J. Cancer., 72: 424-30 (1997)); and Raynes and McAdam, Scand. J. Immunol., 33:657-66 (1991)). Serum amyloid A can be induced in renal tubular epithelial cells, but prior to obtaining the results provided herein, serum amyloid A mRNA had not been associated with CRCC outcome. Finally, CKS2, determined to be upregulated in aggressive CRCC, has been associated with cancer (upregulated in metastatic colon cancer (Li et al., Int. J. Oncol., 24:305-12 (2004)), but its function and significance in CRCC may require further study.

In contrast to a limited number of upregulated genes in aggressive CRCC, there were numerous genes that exhibited decreased mRNA levels relative to non-aggressive CRCC. Several of these genes have been described previously, yet their functional role in CRCC remains unknown. Esophageal cancer-related gene 4 has been identified to be down-regulated in squamous cell carcinoma of the esophagus through hypermethylation of the CpG islands (Lu et al., Int. J. Cancer, 91:288-94 (2001) and Yue et al., World J. Gastroenterol., 9:1174-8 (2003)). The function of this gene is unknown. Likewise, TU3A, a novel gene on chromosome 3p14, was recently found to be deleted in a subset of RCC cell lines (Yamato et al., Cytogenet. Cell. Genet., 87:291-5 (1999)). No studies to date have addressed the biologic or prognostic significance of TU3A in CRCC.

At the present time, there is no standard method for the analysis of microarray data. As described herein, three algorithms were used to identify the best candidate biomarkers common to all three of the algorithms. The fact that all of the candidate biomarkers on the list were validated by the quantitative RT-PCR experiments suggests that the approach for the analysis of microarray data was justified. In addition to gene selection, there are questions regarding normalization in quantitative RT-PCR experiments. To identify genes for normalization of quantitative RT-PCR results, the microarray data was searched for transcripts that displayed minimum variation across the samples. The two transcripts selected by this analysis, EEF1A1 and KPNA6, were confirmed by quantitative RT-PCR to have considerably less variation across the 55 sample validation cohort than the commonly used GapDH and B2M. Furthermore, GapDH and B2M had significantly higher expression levels in CRCC samples than in non-neoplastic kidney. Increased expression of GapDH mRNA in tumor samples is consistent with reports suggesting increased expression of GapDH protein in kidney carcinoma to meet the energy demands of the tumor cells following diminished oxidative phosphorylation in the mitochondria (Cuezva et al., Cancer Res., 62:6674-81 (2002)). Similarly, increased expression of B2M is consistent with reports indicating elevated levels of B2M protein in the serum of renal carcinoma patients (Selli et al., Urol. Res., 12:261-3 (1984)). Comparing the expression levels of EEF1A1 and KPNA6, KPNA6 was chosen for normalization since the expression levels of KPNA6 across the validation samples were more comparable to the expression levels of the selected biomarkers.

CRCC samples were selected based on outcome (good versus poor) and pathologic features. In cases of non-aggressive CRCC with limited follow-up, the SSIGN scoring system was employed to insure that patients considered to have non-aggressive CRCC had a predicted five-year cancer-specific survival of at least 90 percent. In addition, all frozen tissue blocks were reviewed to insure that non-aggressive tumors were all low-grade (nuclear grade 1 and 2). In contrast, patients with CRCC considered aggressive died of disease or developed metastases within 4 years of diagnosis. In addition, review of their tumors revealed predominantly grade 3 and 4. It is possible that this selection process using both outcome and pathologic features improved the ability to identify significant differences in gene expression. In another study of stage I non-small cell cancer of the lung, we were unable to find significant differences in gene expression when cases were selected based only on outcome.

At least two of the transcripts in the list of differentially expressed genes, endomucin (EMCN) and neuropeptide Y receptor Y1 (NPY1R), are believed to be associated with the non-epithelial renal components.

In conclusion, the experimental analyses provided herein identified a panel of potential biomarkers that identified patients with aggressive CRCC. Expression of these genes can provide prognostic information beyond that provided by routine pathologic examination and prognostic scoring systems and algorithms. Inclusion of gene and protein expression data into multivariate analyses that include known prognostic features of CRCC such as TNM stage, nuclear grade, and the presence of necrosis in a large population of patients can be accomplished.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for determining whether a mammal with renal cell carcinoma will have a good or poor outcome, wherein said good outcome comprises living without recurrence of renal cell carcinoma for at least two year following treatment, and wherein said poor outcome comprises dying with renal cell carcinoma within four years of diagnosis or having metastatic renal cell carcinoma within four years of diagnosis, wherein said method comprises determining whether or not said mammal contains renal cell carcinoma cells that express SAA2, HSPC150, xs04h08.x1, IL-8, CKS2, or BIRC3 nucleic acid to an extent greater than the average level of expression exhibited in control cells, wherein said control cells are control renal cell carcinoma cells from a control mammal having said good outcome, wherein the presence of said renal cell carcinoma cells indicates that said mammal will have said poor outcome, and wherein the absence of said renal cell carcinoma cells indicates that said mammal will have said good outcome.

2. The method of claim 1, wherein said mammal is a human.

3. The method of claim 1, wherein said renal cell carcinoma is a clear cell renal cell carcinoma.

4. The method of claim 1, wherein said treatment comprises a nephrectomy.

5. The method of claim 1, wherein said poor outcome comprises dying with renal cell carcinoma within four years of diagnosis.

6. The method of claim 1, wherein said poor outcome comprises having metastatic renal cell carcinoma within four years of diagnosis.

7. The method of claim 1, wherein said method comprises determining whether or not said mammal comprises renal cell carcinoma cells that express SAA2 nucleic acid to an extent greater than the average level of expression exhibited in said control cells.

8. The method of claim 1, wherein said method comprises determining whether or not said mammal comprises renal cell carcinoma cells that express xs04h08.x1 nucleic acid to an extent greater than the average level of expression exhibited in said control cells.

9. The method of claim 1, wherein said method comprises determining whether or not said mammal comprises renal cell carcinoma cells that express IL-8 nucleic acid to an extent greater than the average level of expression exhibited in said control cells.

10. The method of claim 1, wherein said method comprises determining whether or not said mammal comprises renal cell carcinoma cells that express CKS2 nucleic acid to an extent greater than the average level of expression exhibited in said control cells.

11. The method of claim 1, wherein said method comprises determining whether or not said mammal comprises renal cell carcinoma cells that express two or more of the nucleic acids selected from the group consisting of SAA2, HSPC150, xs04h08.x1, IL-8, BIRC3, and CKS2 nucleic acid to an extent greater than the average level of expression exhibited in said control cells.

12. The method of claim 1, wherein said method comprises determining whether or not said mammal comprises renal cell carcinoma cells that express three or more of the nucleic acids selected from the group consisting of SAA2, HSPC150, xs04h08.x1, IL-8, BIRC3, and CKS2 nucleic acid to an extent greater than the average level of expression exhibited in said control cells.

13. The method of claim 1, wherein said method comprises determining whether or not said mammal comprises renal cell carcinoma cells that express SAA2, HSPC150, xs04h08.x1, IL-8, and CKS2 nucleic acid to an extent greater than the average level of expression exhibited in said control cells.

14. The method of claim 1, wherein said determining step comprises measuring the level of SAA2, HSPC150, xs04h08.x1, IL-8, BIRC3, or CKS2 mRNA expressed in said renal cell carcinoma cells.

15. The method of claim 1, wherein said determining step comprises measuring the level of polypeptide expressed from SAA2, HSPC150, xs04h08.x1, IL-8, BIRC3, or CKS2 nucleic acid in said renal cell carcinoma cells.

16. A method for determining whether a mammal with renal cell carcinoma will have a good or poor outcome, wherein said good outcome comprises living without recurrence of renal cell carcinoma for at least two year following treatment, and said poor outcome comprises dying with renal cell carcinoma within four years of diagnosis or having metastatic renal cell carcinoma within four years of diagnosis, wherein said method comprises determining whether or not said mammal comprises renal cell carcinoma cells that express a nucleic acid selected from the group consisting of ECRG4, FLJ32535, PPP2CA, FILIP1, SDPR, SCN4B, PTPRB, 7n51g0.3.x1, TEK, SHANK3, wa07c11.x1, ARG99, SYNPO2, EMCN, DKFZp686P0921_r1, TU3A, NPY1R, MAPT, UI-H-BI4-aqb-d-08-0-UI.s1, LDB2, tn49h09.x1, PDZK3, FLJ22655, tb28a05.x1, FCN3, NX17, CUBN, EPAS1, LOC340024, PLN, ERG, DKFZP564O0823, SLC6A19, and yc17g11.s1 nucleic acid to an extent less than the average level of expression exhibited in control cells, wherein said control cells are control renal cell carcinoma cells from a control mammal having said good outcome, wherein the presence of said renal cell carcinoma cells indicates that said mammal has said poor outcome, and wherein the absence of said renal cell carcinoma cells indicates that said mammal has said good outcome.

17. The method of claim 16, wherein said mammal is a human.

18. The method of claim 16, wherein said renal cell carcinoma comprises clear cell renal cell carcinoma.

19. The method of claim 16, wherein said treatment comprises a nephrectomy.

20. The method of claim 16, wherein said poor outcome comprises dying with renal cell carcinoma within four years of diagnosis.

21. The method of claim 16, wherein said poor outcome comprises having metastatic renal cell carcinoma within four years of diagnosis.

22. The method of claim 16, wherein said method comprises determining whether or not the mammal comprises renal cell carcinoma cells that express two or more of said nucleic acids selected from said group to an extent less than the average level of expression exhibited in said control cells.

23. The method of claim 16, wherein said method comprises determining whether or not said mammal comprise renal cell carcinoma cells that express three or more of said nucleic acids selected from said group to an extent less than the average level of expression exhibited in said control cells.

24. The method of claim 16, wherein said method comprises determining whether or not said mammal comprise renal cell carcinoma cells that express four or more of said nucleic acids selected from said group to an extent less than the average level of expression exhibited in said control cells.

25. The method of claim 16, wherein said method comprises determining whether or not said mammal comprise renal cell carcinoma cells that express five or more of said nucleic acids selected from said group to an extent less than the average level of expression exhibited in said control cells.

26. The method of claim 16, wherein said determining step comprises measuring the level of ECRG4, FLJ32535, PPP2CA, FI LIP1, SDPR, SCN4B, PTPRB, 7n51g0.3.x1, TEK, SHANK3, wa07c11.x1, ARG99, SYNPO2, EMCN, DKFZp686P0921_r1, TU3A, NPY1R, MAPT, UI-H-BI4-aqb-d-08-0-UI.s1, LDB2, tn49h09.x1, PDZK3, FLJ22655, tb28a05.x1, FCN3, NX17, CUBN, EPAS1, LOC340024, PLN, ERG, DKFZP564O0823, SLC6A19, or yc17g11.s1 mRNA expressed in said renal cell carcinoma cells.

27. The method of claim 16, wherein said determining step comprises measuring the level of polypeptide expressed from ECRG4, FLJ32535, PPP2CA, FILIP1, SDPR, SCN4B, PTPRB, 7n51g0.3.x1, TEK, SHANK3, wa07c11.x1, ARG99, SYNPO2, EMCN, DKFZp686P0921_r1, TU3A, NPY1R, MAPT, UI-H-BI4-aqb-d-08-0-UI.s1, LDB2, tn49h09.x1, PDZK3, FLJ22655, tb28a05.x1, FCN3, NX17, CUBN, EPAS1, LOC340024, PLN, ERG, DKFZP564O0823, SLC6A19, or yc17g11.s1 nucleic acid in said renal cell carcinoma cells.

28. A nucleic acid array comprising at least five nucleic acid molecules, wherein each of said at least five nucleic acid molecules comprises a different nucleic acid sequence, and wherein at least 50 percent of said nucleic acid molecules of said array comprise a sequence from a nucleic acid selected from the group consisting of SAA2, HSPC150, xs04h08.x1, IL-8, CKS2, BIRC3, BIRC5, ECRG4, FLJ32535, PPP2CA, FILIP1, SDPR, SCN4B, PTPRB, 7n51g0.3.x1, TEK, SHANK3, wa07c11.x1, ARG99, SYNPO2, EMCN, DKFZp686P0921_r1, TU3A, NPY1R, MAPT, UI-H-BI4-aqb-d-08-0-UI.s1, LDB2, tn49h09.x1, PDZK3, FLJ22655, tb28a05.x1, FCN3, NX17, CUBN, EPAS1, LOC340024, PLN, ERG, DKFZP564O0823, SLC6A19, or yc17g11.s1.

29. The array of claim 28, wherein said array comprises at least ten nucleic acid molecules, wherein each of said at least ten nucleic acid molecules comprises a different nucleic acid sequence.

30. The array of claim 28, wherein said array comprises at least twenty nucleic acid molecules, wherein each of said at least twenty nucleic acid molecules comprises a different nucleic acid sequence.

31. The array of claim 28, wherein each of said nucleic acid molecules that comprise a sequence from a nucleic acid selected from said group comprises no more than three mismatches.

32. The array of claim 28, wherein at least 75 percent of said nucleic acid molecules of said array comprise a sequence from a nucleic acid selected from said group.

33. The array of claim 28, wherein at least 95 percent of said nucleic acid molecules of said array comprise a sequence from a nucleic acid selected from said group.

34. The array of claim 28, wherein said array comprises glass.

35. A method for determining whether a human with clear cell renal cell carcinoma will have a good or poor outcome, wherein said good outcome comprises living without recurrence of clear cell renal cell carcinoma for at least two year following treatment, and wherein said poor outcome comprises dying with clear cell renal cell carcinoma within four years of diagnosis or having metastatic clear cell renal cell carcinoma within four years of diagnosis, said method comprising determining whether or not said human contains clear cell renal cell carcinoma cells that express SAA2, HSPC150, xs04h08.x1, IL-8, and CKS2 nucleic acid to an extent greater than the average level of expression exhibited in control cells, wherein said control cells are control clear cell renal cell carcinoma cells from a control human having said good outcome, wherein the presence of said clear cell renal cell carcinoma cells indicates that said human will have said poor outcome, and wherein the absence of said clear cell renal cell carcinoma cells indicates that said human will have said good outcome.
